(19) United States
(12) Patent Application Publication
(10) Pub. No.: US 2025/0114137 A1
(43) Pub. Date: Apr. 10, 2025

(54) ARTHROSCOPIC DEVICES AND METHODS

(71) Applicant: Biomet Manufacturing, LLC, Warsaw, IN (US)

(72) Inventors: Aaron Germain, San Jose, CA (US); Jan Echeverry, San Jose, CA (US)

(21) Appl. No.: 18/828,664

(22) Filed: Sep. 9, 2024

Related U.S. Application Data
(60) Provisional application No. 63/588,377, filed on Oct. 6, 2023.

(51) Int. Cl. A61B 18/14; A61B 17/00; A61B 18/00

(52) U.S. Cl. CPC A61B 18/148; A61B 18/1445; A61B 2017/00424; A61B 2018/00565; A61B 2018/00577; A61B 2018/00589; A61B 2018/00982; A61B 2018/1457

(57) ABSTRACT

An arthroscopic instrument optionally including: an elongate shaft extending along a longitudinal axis; at least one of a hub or a handpiece coupled to the elongate shaft; an end effector coupled to a distal end of the elongate shaft and having a moveable jaw component; and one or more cameras coupled to the elongate shaft and positioned adjacent the end effector.

### CLAIM OF PRIORITY

This application claims the benefit of U.S. Provisional Patent Application Ser. No. 63/588,377, filed on Oct. 6, 2023, the benefit of priority of which is claimed hereby, and which is incorporated by reference herein in its entirety.

### FIELD OF THE INVENTION

The present invention relates generally to methods and apparatuses for surgical procedures using arthroscopic tissue cutting and removal devices by which anatomical tissues may be cut and removed from a joint or other site.

### BACKGROUND

Arthroscopic surgery is a minimally invasive surgical procedure performed through small incisions in the skin. An arthroscope, which is a small camera, is inserted into the joint to visualize and operate inside the joint. Arthroscopic procedures provide benefits over open surgery such as smaller incisions, less pain, and faster recovery times.

A variety of surgical apparatuses exist for endoscopic cutting and removal of bone including for subacromial decompression, anterior cruciate ligament reconstruction involving notchplasty, and arthroscopic resection of the acromioclavicular joint. Currently, surgeons use arthroscopic shavers and burrs having rotational cutting surfaces to remove tissue in such procedures.

One common arthroscopic procedure is a meniscectomy, which is the removal of torn meniscus cartilage in the knee joint. The meniscus can become damaged due to injury or degeneration and may catch in the knee joint, causing pain and limited mobility. In a meniscectomy procedure, small arthroscopic instruments are inserted into the knee to access, visualize, and remove the damaged portions of the meniscus.

### OVERVIEW

There is a need for a single apparatus that accommodates various functions. Such an apparatus can be inserted into the patient such as via a single port. While individual working ends may have two or more functionalities, such as tissue removal and fluid removal, typically multiple different tools must be utilized during surgery including separate tools to perform imaging of anatomy, perform fluid inflow/outflow and tissue cutting. However, design of such a single apparatus particularly for a meniscectomy is a challenge as regards imaging and providing for both mechanical cutting and radiofrequency (RF) coagulation and ablation.

The present inventor has developed improved surgical apparatuses and methods, such as with the capability to be inserted into the anatomy such as the knee joint via a single access port and having a combined capability to perform imaging, fluid inflow and removal, along with endoscopic tissue cutting and coagulation using mechanical or RF mechanisms. The present inventor has also developed a surgical apparatus with further capabilities including an apparatus that can perform various imagining functions to change the field of view, can illuminate the surgical site, can remove tissue debris generated from the cutting or other processes, provide irrigation, etc. The present inventor contemplates the surgical apparatuses disclosed herein can reduce costs by eliminating multiple surgical tools, reduce patient discomfort by having only a single access port, reduce surgical complexity and reduce surgical time among other benefits.

### 2. Description of the Background Art

Relevant commonly owned patent publications include: U.S. Pat. Nos. 11,065,023; 11,172,953; US 2018-0303509; US 2019-0008541; US 2019-0059983; US 2019-0134279; US 2019-0021788; US 2018-0317957; US 2019-0008538; US 2019-0083121; US 2018-0263649; US 2017-0290602 and US 2019-0015151, the full disclosures of each of which are incorporated herein by reference.

The following, non-limiting examples, detail certain aspects of the present subject matter to solve the challenges and provide the benefits discussed herein, among others.

Example 1 is a method for performing arthroscopic meniscectomy optionally comprising: accessing a joint of a patient; passing an arthroscopic instrument into the joint to a torn meniscus of the joint; visualizing at least a portion of an end effector of the arthroscopic instrument with one or more cameras coupled to the arthroscopic instrument adjacent the end effector; and treating the meniscus with the end effector.

In Example 2, the subject matter of Example 1 optionally includes, wherein treating the meniscus with the end effector includes mechanically cutting the meniscus with actuation of a moveable jaw component.

In Example 3, the subject matter of Examples 1-2 optionally includes, wherein treating the meniscus with the end effector includes providing radiofrequency energy to the meniscus.

In Example 4, the subject matter of Example 3 optionally includes, wherein the treating the meniscus with the radiofrequency energy includes moving a position of an electrode with respect to a stationary portion of the end effector.

In Example 5, the subject matter of Examples 1-4 optionally includes, wherein treating the meniscus the end effector includes providing a fluid inflow and outflow to the end effector.

In Example 6, the subject matter of Examples 1-5 optionally includes, wherein the visualizing includes illuminating the at least the portion of the end effector with a light source coupled to the arthroscopic instrument adjacent the end effector.

In Example 7, the subject matter of Examples 1-6 optionally includes, wherein accessing the joint of the patient is through a single access port that receives a shaft of the arthroscopic instrument.

In Example 8, the subject matter of Examples 1-7 optionally includes, wherein the one or more cameras are positioned with a field of view that includes the at least the portion of the end effector and a target tissue including the meniscus.

In Example 9, the subject matter of Examples 1-8 optionally includes, moving the one or more cameras from a first position with a first field of view that includes the at least the portion of the end effector to a second position with a second field of view that differs from the first field of view.

In Example 10, the subject matter of Examples 1-9 optionally includes, orienting the end effector and the one or more cameras prior to visualizing and treating.

Example 11 is an arthroscopic instrument optionally comprising: an elongate shaft extending along a longitudinal axis; at least one of a hub or a handpiece coupled to the elongate shaft; an end effector coupled to a distal end of the elongate shaft and having a moveable jaw component; and one or more cameras coupled to the elongate shaft and positioned adjacent the end effector.

In Example 12, the subject matter of Example 11 optionally includes, wherein the moveable jaw component includes an electrode at a distal end portion thereof.

In Example 13, the subject matter of Examples 11-12 optionally includes, an electrode coupled to a suction tube of the end effector.

In Example 14, the subject matter of Example 13 optionally includes, wherein the suction tube is configured to be moveable proximal-distal along the end effector including being configured to be moveable to a position at or distal of a distal tip of the end effector.

In Example 15, the subject matter of Examples 11-14 optionally includes, wherein the one or more cameras are part of a visualization assembly that is configured to be moveable from a first position that has a first field of view including at least a portion of the end effector to a second position with a second field of view that differs from the first field of view.

In Example 16, the subject matter of Example 15 optionally includes, wherein the visualization assembly is at least one of rotatable relative to the end effector or extendible and retractable relative to the end effector.

In Example 17, the subject matter of Examples 15-16 optionally includes, wherein the visualization assembly includes a light source.

In Example 18, the subject matter of Examples 11-17 optionally includes, wherein the shaft includes one or more ports configured for inflow or outflow of a fluid proximal of the end effector.

In Example 19, the subject matter of Examples 11-18 optionally includes, wherein the one or more cameras and the end effector are angled relative to the longitudinal axis of the elongate shaft.

In Example 20, the subject matter of Examples 18-19 optionally includes, an articular joint connecting the one or more cameras and the end effector to the elongate shaft, wherein the articular joint is configured to move the one or more cameras and the end effector relative to the longitudinal axis.

Example 21 is an arthroscopic instrument for performing a meniscectomy optionally comprising: an elongate shaft extending along a longitudinal axis; at least one of a hub or a handpiece coupled to the elongate shaft; an end effector coupled to a distal end of the elongate shaft and having a moveable jaw component; and a visualization assembly positioned adjacent the end effector and including one or more cameras with a field of view that includes, at least a portion of the end effector and a target tissue including a torn meniscus.

In Example 22, the subject matter of Example 21 optionally includes, wherein the end effector includes an electrode for delivering radiofrequency therapy, wherein the electrode is coupled to one of: the moveable jaw component, a stationary jaw component or a suction tube.

In Example 23, the subject matter of Example 22 optionally includes, wherein the suction tube is configured to be moveable proximal-distal along the end effector including being configured to be moveable to a position at or distal of a distal tip of the end effector.

In Example 24, the subject matter of Examples 21-23 optionally includes, wherein the visualization assembly that is configured to be moveable from a first position with the field of view to a second position with a second field of view that differs from the first field of view.

In Example 25, the subject matter of Example 24 optionally includes, wherein the visualization assembly is at least one of rotatable relative to the end effector or extendible and retractable relative to the end effector.

In Example 26, the subject matter of Examples 24-25 optionally includes, wherein the visualization assembly includes a light source.

In Example 27, the subject matter of Examples 21-26 optionally includes, wherein the shaft includes one or more ports configured for inflow or outflow of a fluid proximal of the end effector.

In Example 28, the subject matter of Examples 21-27 optionally includes, wherein the visualization assembly and the end effector are one of: angled relative to the longitudinal axis of the elongate shaft or rotatable relative to the longitudinal axis of the elongate shaft.

Example 29 is at least one machine-readable medium including instructions that, when executed by processing circuitry, cause the processing circuitry to perform operations to implement of any of Examples 1-28.

Example 30 is an apparatus comprising means to implement of any of Examples 1-28.

Example 31 is a system to implement of any of Examples 1-28.

Example 32 is a method to implement of any of Examples 1-28.

In Example 33, the instruments, methods or systems of any one or any combination of Examples 1-32 can optionally be configured such that all elements or options recited are available to use or select from.

### BRIEF DESCRIPTION OF THE DRAWINGS

Various embodiments of the present invention will now be discussed with reference to the appended drawings. It should be appreciated that the drawings depict only typical embodiments of the invention and are therefore not to be considered limiting in scope.

FIG. 1A is a side view of an arthroscopic instrument with multiple operational functionalities according to an example of the present disclosure.

FIG. 1B is a cross-sectional view of the arthroscopic instrument of FIG. 1A.

FIG. 1C is a side view of a first side of the arthroscopic instrument of FIG. 1A.

FIG. 1D is a side view of a second side of the arthroscopic instrument of FIG. 1A.

FIG. 2 is an enlarged perspective view of a distal (working) end of the arthroscopic instrument of FIGS. 1A-1D showing various components thereof according to an example of the present disclosure.

FIG. 3 is an cross-sectional view of the distal (working) end of the arthroscopic instrument of FIGS. 1A-2 according to another example of the present disclosure.

FIG. 4 is a side view of an arthroscopic instrument with multiple operational functionalities according to another example of the present disclosure.

FIG. 4A is an enlarged perspective view of the distal (working) end of the arthroscopic instrument of FIG. 4 according to another example of the present disclosure.

FIG. 4B is a first side view of the distal (working) end of the arthroscopic instrument of FIG. 4 according to another example of the present disclosure.

FIG. 4C is a second side view of the distal (working) end of the arthroscopic instrument of FIG. 4 according to another example of the present disclosure.

FIG. 5A is a schematic view of the distal (working) end of the arthroscopic instrument with a visualization assembly in a first position according to an example of the present disclosure.

FIG. 5B is a schematic view of the distal (working) end of the arthroscopic instrument of FIG. 5A with the visualization assembly rotated to a second position according to an example of the present disclosure.

FIG. 6A is a schematic view of the distal (working) end of the arthroscopic instrument with the visualization assembly in the process of being translated from a first position toward a second position according to an example of the present disclosure.

FIG. 6B is a schematic view of the distal (working) end of the arthroscopic instrument of FIG. 6A with the visualization assembly in the second position according to an example of the present disclosure.

FIG. 7A is a schematic view of the distal (working) end of the arthroscopic instrument with a suction apparatus with an electrode in the process of being translated from a first posterior position toward a second distal position according to an example of the present disclosure.

FIG. 7B is a schematic view of the distal (working) end of the arthroscopic instrument of FIG. 7A with the suction apparatus and the electrode in the second distal position according to an example of the present disclosure.

FIG. 8 is a schematic view of the distal (working) end of the arthroscopic instrument with a movable active electrode according to an example of the present disclosure.

FIGS. 9A and 9B are schematic views of the distal (working) end of the arthroscopic instrument with the distal (working) end having a fixed angle relative to a longitudinal axis of the elongate shaft according to an example of the present disclosure.

FIG. 10 is a schematic view of the distal (working) end of the arthroscopic instrument with the distal (working) end being configured to articulate to a desired angle relative to a longitudinal axis of the elongate shaft according to an example of the present disclosure.

FIG. 11 is an exploded view of an arthroscopic cutting system that includes an arthroscopic instrument having reusable handpiece with a detachable single-use probe according to an example of the present disclosure.

FIG. 12 is a cross-sectional view of the detachable single-use probe of the FIG. 11 showing an actuation mechanism for moving the jaws and optionally moving the visualization assembly with the actuation mechanism according to an example of the present disclosure.

FIG. 13 is a flow chart of a method for performing arthroscopic meniscectomy according to an example of the present application.

### DETAILED DESCRIPTION OF THE INVENTION

The present invention relates to arthroscopic instruments that have various functions. Several embodiments of the devices will now be described to provide an overall understanding of the principles of the form, function and methods of use. In general, the present disclosure provides for electrosurgical devices that can be used as arthroscopic instruments including for a meniscectomy. However, the electrosurgical devices disclosed herein are not limited to meniscectomy and can be utilized in other surgical procedures. The electrosurgical devices described herein can perform more than one surgical function. Thus, the electrosurgical devices can be configured for anatomy visualization, fluid inflow and outflow, coagulation and/or cutting bone such as of soft tissue, meniscal tissue, etc. using mechanical mechanisms and/or RF energy. The arthroscopic instruments can be entirely disposable or can be an assembly with a disposable probe configured for detachable coupling to a non-disposable reusable handpiece. Functions contemplated include vacuum aspiration of fluids including tissue debris through a shaft of the probe and outwardly through the reusable handpiece without interfering with the electrical and/or mechanical operation of the surgical system to deliver radiofrequency (RF) current to the probe. This description of the general principles of this invention is not meant to limit the inventive concepts in the appended claims.

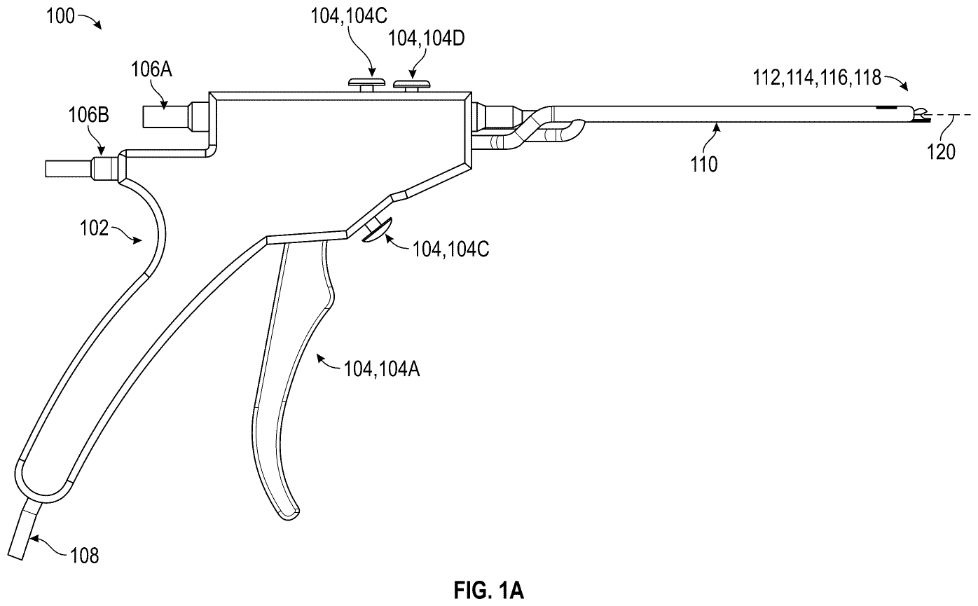

FIGS. 1A-1D show an arthroscopic instrument 100. As shown in FIGS. 1A and 1B, the arthroscopic instrument 100 can include a handpiece 102, a plurality of actuators 104A, 104B, 104C and 104D, a plurality of lumens 106A and 106B, a cable assembly 108 and one or more elongate shafts 110. The arthroscopic instrument 100 has a working end 112 that carries a plurality of end effectors 114 and a visualization assembly 116. The plurality of end effectors 114 can include a plurality of tissue cutting features 118 such as a moveable jaw(s) and RF electrodes configured for use in many arthroscopic surgical applications, including but not limited to treating tissue in shoulders, knees, hips, wrists, ankles and the spine.

The handpiece 102 and one or more elongate shafts 110 can be coupled together. The one or more elongate shafts 110 can extend along a longitudinal axis 120. The working end 112 can be coupled to or can be part of the one or more elongate shafts 110 as shown and described subsequently. The one or more elongate shafts 110 can be somewhat flexible or rigid as desired and can house various components including portions of the plurality of lumens 106A and 106B and the cable assembly 108 that can extend from the handpiece 102 to the distal end 112 as further discussed. Thus, the plurality of lumens 106A and 106B and at least part of the cable assembly 108 can extend through the handpiece 102 and along the one or more elongate shafts to the working end 112. The one or more elongate shafts 110 can comprise tube(s) or outer sleeve(s) with components such as wires, flow channels, additional shafts, and the like passing therethrough. The one or more elongate shafts 110 extend from the handpiece 102 (located at a proximal end of the one or more elongate shafts 110) to the distal end 112. The one or more elongate shafts 110 can be coupled in a fixed manner or moveable manner to the handpiece 102. One or more of the plurality of actuators 104A, 104B, 104C and 104 can be utilized in some examples to actuate movement or features of the plurality of end effectors 114, the visualization assembly 116, fluid inflow/outflow or other features as discussed herein. The handpiece 102 and/or the one or more elongate shafts 110 can be formed of an injection molded plastic, for example. Alternatively, the one or more elongate shafts 110 can be insert molded into the handpiece 102. One or more components or features can pass through one or more elongate shafts 110 including to provide irrigation, fluid outflow, visualization, RF energy to the electrodes or the like. The number of plurality of actuators 104A, 104B, 104C and 104 shown are purely exemplary and all such actuators need not be utilized in some examples of the present application.

The handpiece 102 can be operatively coupled by the cable assembly 108 to an energy source and/or a controller (not shown) which can control or aid with at least some of the functions such as visualization implemented by the instrument 100. The cable assembly 108 can be coupled to various features including the RF electrodes (part of the cutting features 118), the visualization assembly 116 and the plurality of actuators 104A, 104B, 104C and 104D. The controller, for example, can operate and control some or all functionality, which includes controlling the RF source, the flow inducing device, visualization assembly 116, and the negative pressure source which can aspirate fluid including tissue debris to a collection reservoir. The suction lumen 106A can be in communication with a negative pressure source (not shown). The inflow lumen 106B can be in communication with flow inducing device (not shown) and is configured to allow for flow to the distal end 112 of a fluid such as for application of an irrigating fluid (e.g., saline) utilized during operation of the instrument 100. The cable assembly 108 can be in electrical communication with an RF or other power source. The plurality of actuators 104A, 104B, 104C and 104 on the handpiece 102 can be used in concert with the controller or other on device control unit to implement visualization changes, actuate mechanical cutting, select operating modes, such as current strength for RF, flow control or the like.

FIG. 1B shows the visualization assembly 116 can include a microchip 124 such as a printed circuit board (PCB) located on the instrument 100 itself for control of light and/or camera function separate from an off-instrument controller. The microchip 124 can be electronically coupled to the actuators 104C and 104D, for example. Thus, the arthroscopic instrument 100 can have feature(s) such as light and camera that are controlled by a system on chip architecture, for example. The arthroscopic instrument 100 can be operated in different RF modes. One mode can deliver RF current in a cutting waveform to thereby create a plasma that ablates tissue. This mode can be controlled by the controller (not shown) or the microchip 124. In another RF mode, the controller or the microchip 124 can include an algorithm that controls RF current in a coagulation waveform that can be delivered to the electrodes for coagulation of tissue. Actuation of the RF mode (on, off, coagulate, ablate) can be controlled with the actuator 104B, for example. The actuator 104A can be coupled to a hub portion 125 (FIG. 1B) coupled to internal components of the one or more elongate shafts 110 and on to the tissue cutting features 118 such as the jaw as further discussed below. The actuator 104A can be configured to linearly actuate (push and pull) the hub portion 125 and parts of the one or more elongate shafts 110 to manipulate the tissue cutting features 118, such as the jaw, for example.

FIGS. 1C and 1D show the arthroscopic instrument 100 from various sides. It should be noted that in FIGS. 1C-1D, the one or more elongate shafts 110 include a first shaft 110A and a second shaft 110B. The first shaft 110A and the second shaft 110B can be positioned to extend generally parallel with one another. The first shaft 110A can include features of one or more of the plurality of lumens 106A and 106B (FIGS. 1A and 1B), the RF electronics and actuators for mechanical cutting features. The second shaft 110B can house parts of the visualization assembly 116. According to some examples some of the plurality of lumens 106A and/or 106B (FIGS. 1A and 1B) can be housed by the second shaft 110B. The first shaft 110A and the second shaft 110B can extend generally in parallel with one another and can each have the distal end 112 that terminates in close proximity (e.g., terminates within 10 mm or less) of one another. Although FIGS. 1A-1D illustrate the use of two separate shafts, other examples contemplate a single shaft is utilized.

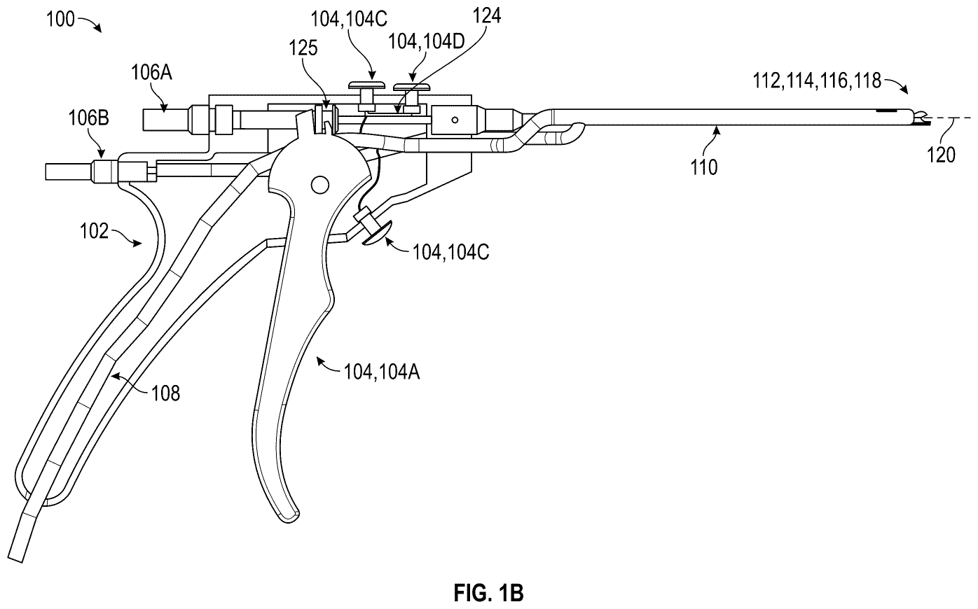

FIG. 2 shows the distal (working) end 112 of the arthroscopic instrument 100. In particular, FIG. 2 shows the first shaft 110A coupled to the plurality of end effectors 114 including the tissue cutting features 118. The end effectors 114 can be coupled to or can be adjacent the distal end 112 of the first shaft 110A and/or the second shaft 110B. As shown in FIG. 2, the visualization assembly 116 can be coupled to the second shaft 110B. The visualization assembly 116 can include a light source 126 and a camera 128. The distal end 112 of the second shaft 110B can additionally define a port 130 that is a terminus for the inflow lumen 106B. The tissue cutting features 118 can be coupled to the first shaft 110A. The tissue cutting features 118 can include a moveable jaw 132, a second jaw 134, an active electrode 136 and a passive electrode 138. The distal end 112 can additionally have a port 140 for the suction lumen 106A. A combined width W of the distal end 112 can be about twice that of a height of the distal end 112. For example, the combined width can be about 6 mm while the height can be about 3 mm. This stacked width configuration works well in the arthroscopic knee environment where the vertical space between the bones is limited but the lateral space is greater. However, other configurations for the distal end 112 are contemplated.

The light source 126 can include one or more light emitting diodes (LEDs) or other illumination components. The LED can be an on-chip controlled device with the chip being at the distal end 112 or at another location on the instrument. This allows the LED to be controlled via actuator or other interface on the arthroscopic instrument 100, for example. The camera 128 can be an on-chip controlled device with the chip being at the distal end 112 or on the instrument at another location. This allows the camera 128 to be controlled via actuator or other interface on the arthroscopic instrument 100, for example. For example, the camera 128 can utilize Complementary Metal-Oxide Semiconductor Active Pixel Sensors (CMOS-APS). The CMOS-APS are configured for sensing at an infrared wavelength range, a visual light wavelength range or another wavelength range as desired. The light source 126 and/or the camera 128 can be angled relative to a longitudinal axis of the second shaft 110B and/or the first shaft 110A so as to be pointed toward a longitudinal axis of the first shaft 110. Due to such angulation the light source 126 and/or the camera 128 can have a field of view that includes at least a part of the tissue cutting features 118. This arrangement allows the light source 126 and the camera 128 to cast light and view into the moveable jaw 132 and/or the second jaw 134 and beyond the moveable jaw 132 and/or the second jaw 134 so that the target tissue can be visualized before and during cutting and/or coagulation. Thus, the visualization assembly 116 can have the field of view that includes at least a portion of the end effector 114 and a target tissue according to some examples. The angulation of the light source 126 and/or the camera 128 can be between 5 degrees and 45 degrees relative to the longitudinal axis of the second shaft 110B, for example.

The moveable jaw 132 can be pivotally moveable relative to the second jaw 134 from an open position to a closed position and back again. The moveable jaw 132 is shown in the open position in FIG. 2. The moveable jaw 132 can be pivotally connected to the first shaft 110A or other stationary part of the distal end 112. The moveable jaw 132 can be driven by actuation of an interior connector such as a wire or shaft (not shown) or other component coupled to the hub portion 125 of FIG. 1B. The moveable jaw 132 can be suitably shaped such as by having one or more sharps (e.g., cutting edge(s), teeth, etc.). The moveable jaw 132 cutting surface(s) can be generally U-shaped capable of biting off or otherwise cutting suitably shaped portions of tissue. The moveable jaw 132 can be movable into close proximity and/or engagement with the second jaw 134. The second jaw 134 can be stationary. However, according to other examples the second jaw 134 can move such as in concert with the moveable jaw 132. The second jaw 134 can be slightly larger in width so as to receive the moveable jaw 132 at least partially therein when the moveable jaw 132 is in the closed position. The second jaw 134 can be suitably shaped such as by having one or more sharps (e.g., cutting edge(s), teeth, etc.). The second jaw 134 cutting surface(s) can be generally U-shaped configured to work in concert with the moveable jaw 132 in cutting suitably shaped portions of tissue.

The active electrode 136 can be coupled to the second jaw 134 or can be in close proximity thereto. The active electrode 136 can be insulated from the second jaw 134. The active electrode 136 can be located at a distalmost tip of the distal end 112, for example. The passive electrode 138 can be spaced proximally from the active electrode 136 and can be part of or coupled to the first shaft 110A proximal of the distal end 112, for example. The active electrode 136 can be almost entirely surrounded by insulating material (e.g., ceramic, etc.). The port 140 for the suction lumen 106A can be defined by an interior portion of the moveable jaw 132 and can be selectively cut when the moveable jaw 132 is in the closed position.

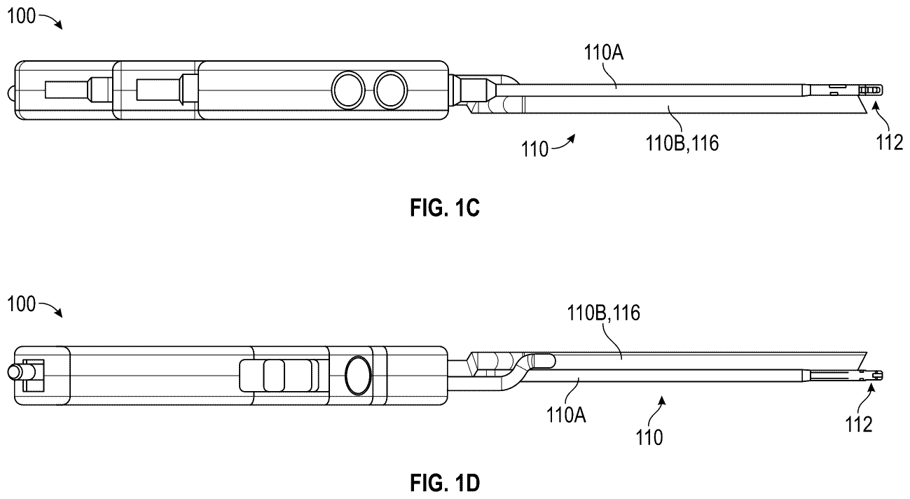

FIG. 3 is a cross-section through the first shaft 110A and illustrates part of the first shaft 110A, the second shaft 110B, the distal end 112 and the plurality of end effectors 114. As shown in FIG. 3, the visualization assembly 116 at the distal end 112 is angled to have the field of view that includes the tissue cutting features 118 as discussed previously. FIG. 3 shows the moveable jaw 132, the second jaw 134, the active electrode 136, the passive electrode 138, and the suction lumen 106A. FIG. 3 additionally shows a connector 142, insulated RF wire 144, a first pivot pin 146 and a second pivot pin 148.

The connector 142 can be a smaller shaft, wire or other component within the cannulated first shaft 110A. The connector 142 can be moveable to translate generally proximal-distal (via actuation of the actuator 104A and the hub portion 125 of FIG. 1B) to actuate the moveable jaw 132. The connector 142 can be coupled to the moveable jaw 132 via the first pivot pin 146. The insulated RF wire 144 can extend within and in some cases can partially define the suction lumen 106A at the distal end 112 and can extend out to the active electrode 136 at the second jaw 134. The moveable jaw 132 can be connected to the second jaw 134 via the second pivot pin 148.

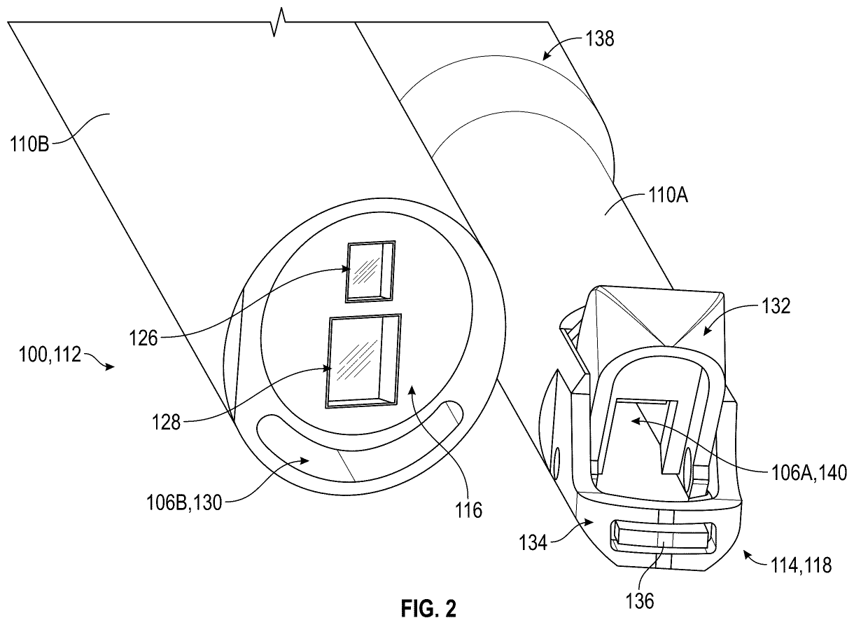

FIGS. 4-4C show an arthroscopic instrument 200 of similar construction to that of the arthroscopic instrument 100 discussed previously with regard to FIGS. 1A-3. FIGS. 4A-4C show a distal end 212 that can differ somewhat from the previous embodiment. As shown in FIGS. 4A and 4C, a first shaft 210A of the arthroscopic instrument 200 proximal of the distal end 212 and a plurality of end effectors 214 includes a plurality of ports 230 for inflow of fluid from an inflow lumen (not shown) to the surgical site.

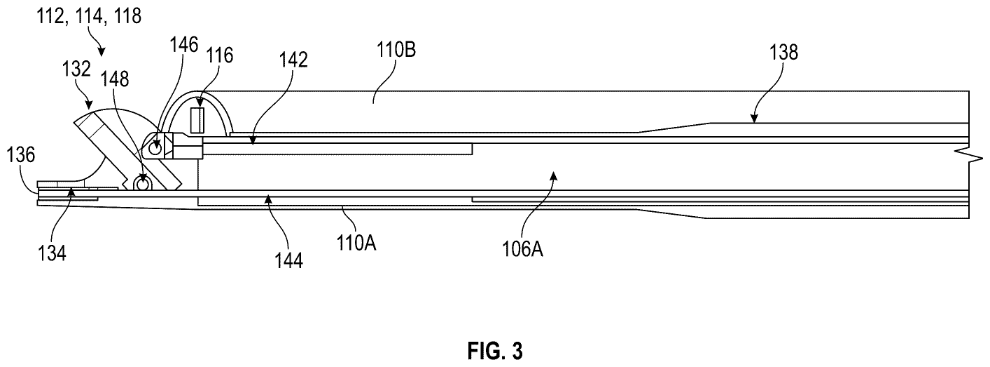

FIGS. 5A and 5B show a distal (working) end 312 of an arthroscopic instrument 300. The arthroscopic instrument 300 can have a construction similar to that of the arthroscopic instrument 100 discussed previously with regard to FIGS. 1A-3. As shown in FIGS. 5A and 5B, a visualization assembly 316 can differ in that it can be moveable to alter a field of view. In particular, the visualization assembly 316 is configured to be moveable from a first position (FIG. 5A) that has a first field of view including at least a portion of an end effector 314 to a second position (FIG. 5B) with a second field of view that differs from the first field of view. In particular, the visualization assembly 316 is rotatable from a first position (shown in FIG. 5A) to a second position (shown in FIG. 5B). As shown in FIGS. 5A and 5B, a light source 326 and a camera 328 of the visualization assembly 316 is rotatable such by one of the shafts to any of: 0 degrees, 90 degrees, 180 degrees or 360 degrees relative to the end effector 314. This rotatable configuration can allow for a larger and/or different field of view. In the second position of FIG. 5B, the camera 328 can be in an endoscope position, having the field of view that does not include the end effector(s) 314 such as the jaws but includes adjacent tissue.

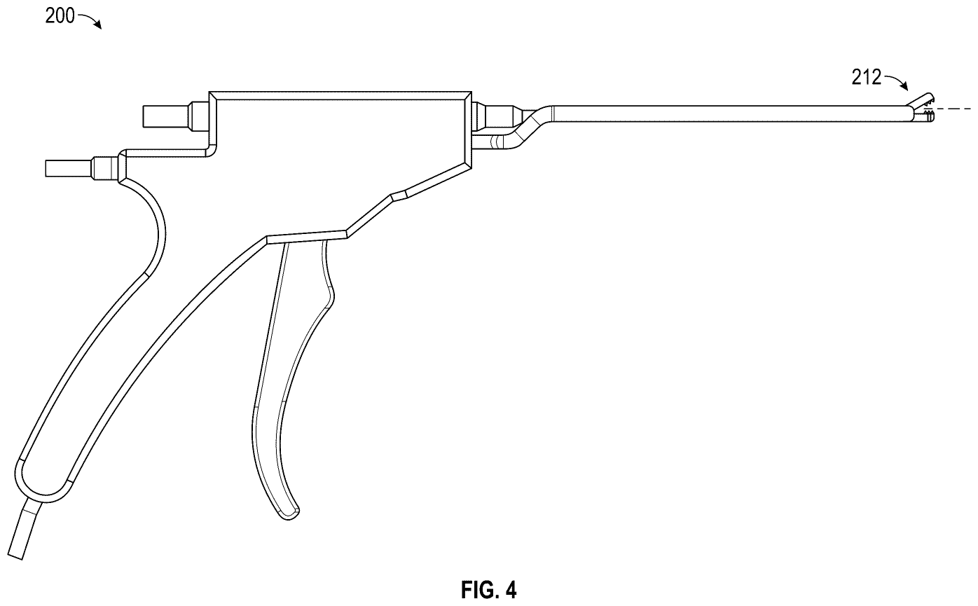

FIGS. 6A and 6B show a distal (working) end 412 of an arthroscopic instrument 400. The arthroscopic instrument 400 can have a construction similar to that of the arthroscopic instrument 100 discussed previously with regard to FIGS. 1A-3. As shown in FIGS. 6A and 6B, a visualization assembly 416 can differ in that it can be moveable to alter a field of view. In particular, the visualization assembly 416 is configured to be moveable from a first position (FIG. 6A) that has a first field of view including at least a portion of an end effector 414 to a second position (FIG. 6B) with a second field of view that differs from the first field of view. In particular, the visualization assembly 416 is linearly extendible with movement of a shaft from a first position (shown in FIG. 6A) to a second position (shown in FIG. 6B). As shown in FIGS. 6A and 6B, a light source 426 and a camera 428 of the visualization assembly 416 can have an altered field of view that does not include some (or all of) end effectors 414 such as the jaws but includes adjacent tissue. The second position of FIG. 6B (or FIG. 5B) could be utilized when cutting of tissue is not needed and can be used during a diagnostic phase of a procedure prior to cutting.

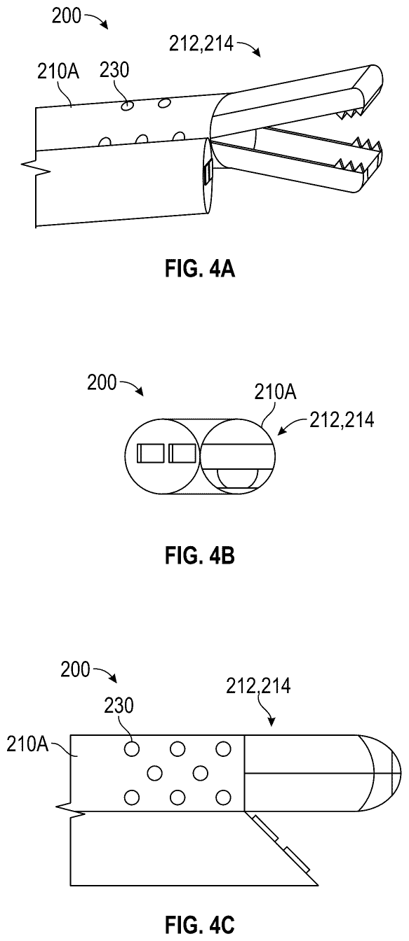

FIGS. 7A and 7B show a distal (working) end 512 of an arthroscopic instrument 500. The arthroscopic instrument 500 can have a construction similar to that of the arthroscopic instrument 100 discussed previously with regard to FIGS. 1A-3. As shown in FIGS. 7A and 7B, a suction tube 502 that defines a suction lumen can be configured to be moveable proximal-distal along or adjacent an end effector 514 (such as the mechanical jaw(s)). This can include being configured to be moveable to a position at or distal of a distal tip of the end effector 514 as shown in FIG. 7B. Thus, the suction tube 502 can be extendible from and retractable into the end effector 514. An active electrode 536 can be positioned at a distal tip of the suction tube 502.

The configurations of FIGS. 7A and 7B can be used for various purposes. In the retracted position of FIG. 7A, the plasma created adjacent the active electrode 536 can be ignited to cut tissue and prevent clogging of the suction lumen. In the extended position of FIG. 7B, the active electrode 536 can be used for coagulation or to contact, contour and/or shape tissue with use of RF.

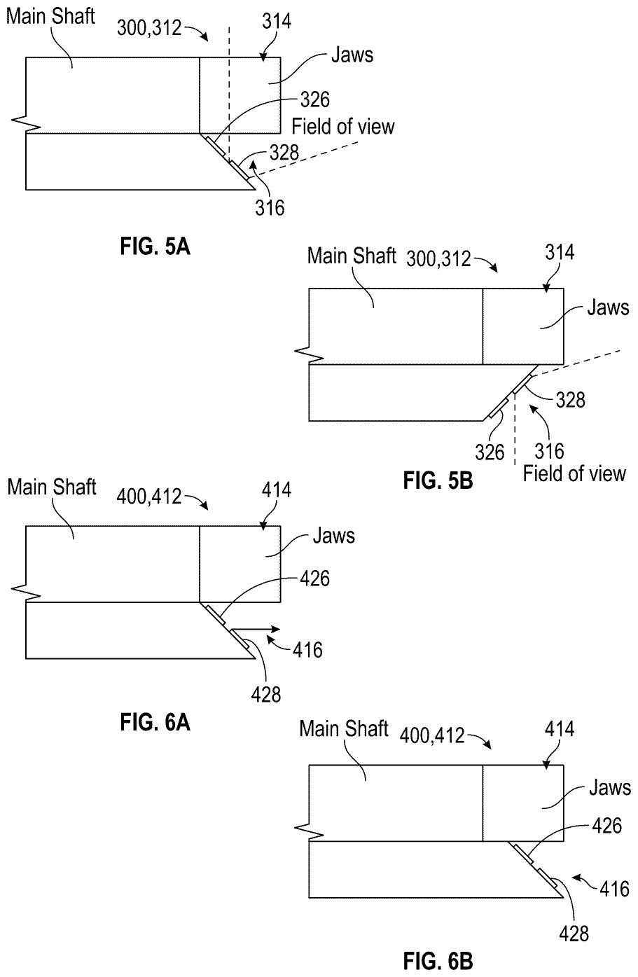

FIG. 8 shows a distal (working) end 612 where the active electrode 636 is mounted to a moveable jaw 632 and insulated therefrom. The passive or return electrode 638 can be part of a second stationary jaw 634. The active electrode 636 can be paired with insulating material such as ceramic that forms a parts of the first jaw 632 and the second jaw 634, for example.

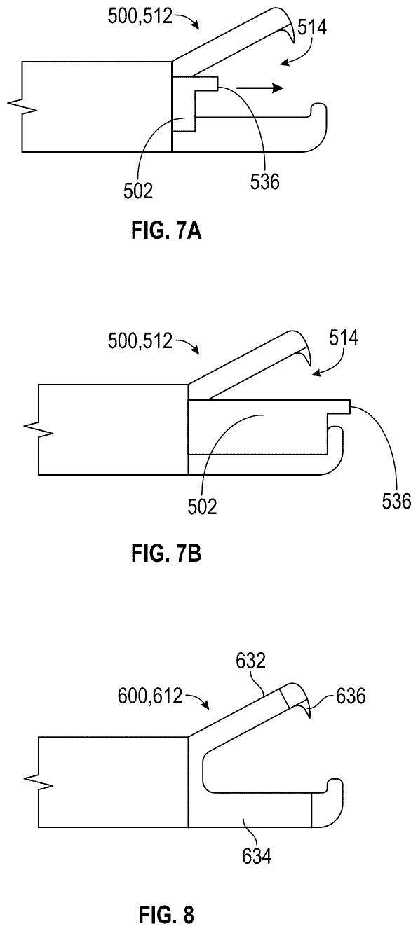

FIGS. 9A and 9B show a distal (working) end 712 where at least the end effector(s) 714 are angled relative to a longitudinal axis of shaft(s) 710A and 710B. Additionally, a visualization assembly 716 can be angled relative to a longitudinal axis of the shaft(s) 710A and 710B. Such angles are fixed in FIGS. 9A and 9B and can be anything from 0.1 degrees angulation to 90 degrees angulation.

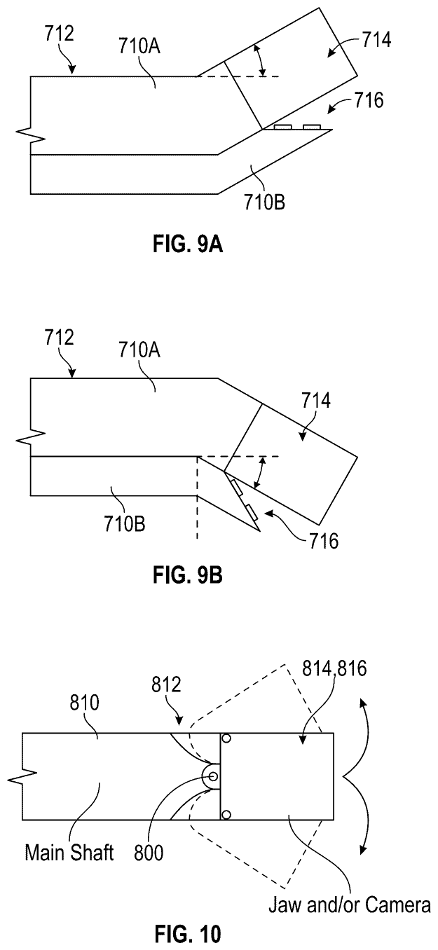

FIG. 10 shows a distal (working) end 812 where the at least the end effector(s) 814 can be angled by selective articulation relative to a longitudinal axis of a shaft(s) 810. As an example, the end effector(s) 814 and a visualization assembly 816 or the shaft(s) 810 can include an articular joint 800. The articular joint 800 can connect one or more cameras of the visualization assembly 816 and the end effector to the elongate shaft (shaft(s) 810). The articular joint 800 can be configured to move the one or more cameras (part of the visualization assembly 816) and the end effector 814 relative to a longitudinal axis of the elongate shaft. Additionally, a visualization assembly 816 can be angled by articulation relative to a longitudinal axis of the shaft(s) 810.

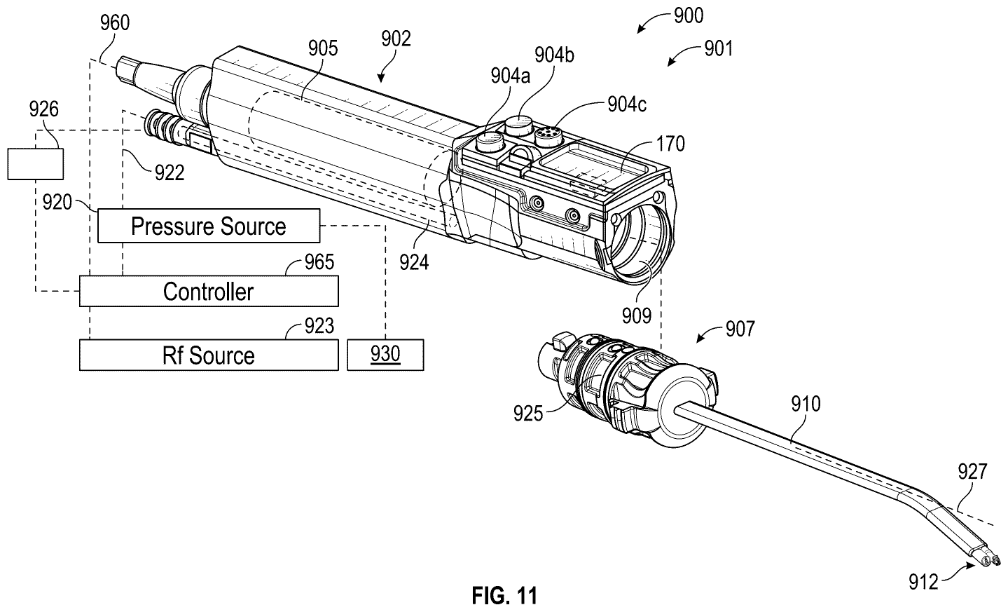

FIG. 11 shows an arthroscopic system 901 of the present invention provides an arthroscopic instrument 900 having a handpiece 902 with motor drive 905 and a probe 907. The arthroscopic instrument 900 replaces the mechanical handle and “grasper” mechanism (actuator 104A of FIG. 1) with a motorized mechanism which is driven by the handpiece 902. The probe 907 with a hub portion 925 that can be received by receiver or bore 909 in the handpiece 902. The probe 907 has a working end 912 that carries the jaw(s) and RF electrodes as discussed previously.

The probe 907 is attachable to and detachable from the handpiece 902. The probe 907 has a plurality of shafts 910 extending along longitudinal axis 927. A distal portion of the one or more shaft(s) 110 including the distal end 112 can be angled (e.g., by 15 degrees, or the like) relative to the longitudinal axis 927.

In FIG. 11, the handpiece 902 is operatively coupled by electrical cable 960 to a controller 965 which can control the motor drive 905, communication with a pressure source 920, and communication with the RF source 923. Actuator buttons 904a, 904b, 904c, etc. on the handpiece 902 can be used to select operating modes, such as current strength for RF, motor speed, flow control, illumination control, video control, image capture or the like. It can be understood from FIG. 11 that the system 901 and the handpiece 902 can be configured for use with various disposable probes.

The system 901 also includes the pressure source 920 such as a negative pressure source coupled to aspiration tubing 922 which communicates with a flow channel 924 in handpiece 902 and can cooperate with one or more tubes of the probe 907. The system 901 includes the RF source 923 which can be connected to an electrode arrangement of the probe 907. The system 901 can include flow inducing device 926 such as a pump, positive pressure source or the like that passes in fluid communication to the handpiece 902 and to the distal end 912. The flow inducing device 926 (optionally controlled by the controller 965) can allow for flow to the distal end 112 of a fluid such as for application of an irrigating fluid (e.g., saline) utilized during operation of the arthroscopic instrument 900. The controller 965 and microprocessor therein together with control algorithms are provided to operate and control all functionality, which includes controlling the motor drive 905, the RF source 923, the flow inducing device 926, illuminating device, camera, and the negative pressure source 920 which can aspirate fluid including tissue debris to collection reservoir 930.

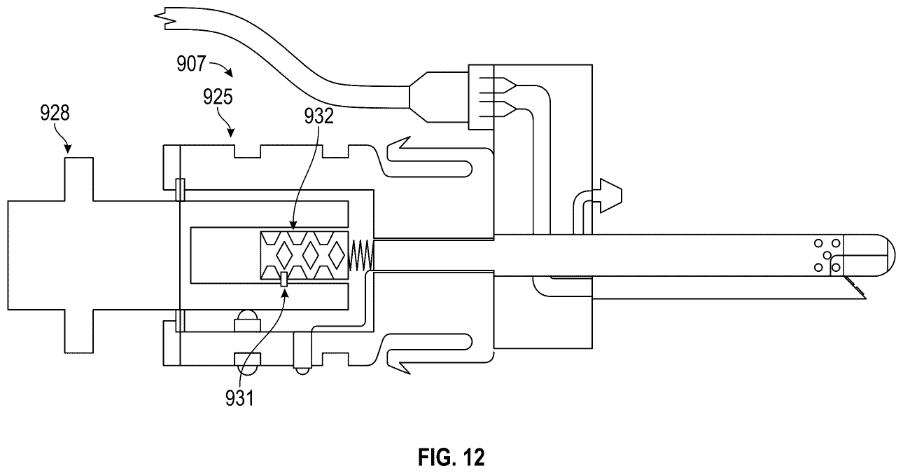

FIG. 12 shows the probe 907 of FIG. 11 including the hub portion 925. The hub portion 925 can include a drive coupler 928 that connects the handpiece 902 (FIG. 11) to the motor drive 905 (FIG. 11). The hub portion 925 can be rotationally drive and rotates a cam follower 931 around a double threaded screw 932. The double threaded screw 932 has both left and right handed thread. The cam follower 931 can follow the right handed thread which moves an inner connector distal (closing the jaw for example). At a bottom of the thread, the cam follower 931 going up a ramp, which can position the cam follower 931 left handed thread. Movement on the left handed thread will retract the connector (moving it proximal and closing the jaw(s) for example). In this manner, the jaw(s) can be selectively driven open and closed with the drive coupler 928 as driven by the motor drive 905. A spring coupled to the connector can follow movement of the jaw(s) and can become compressed with closing movement of the jaw(s) to ensure tolerance and closing force of the jaw(s) is as desired.

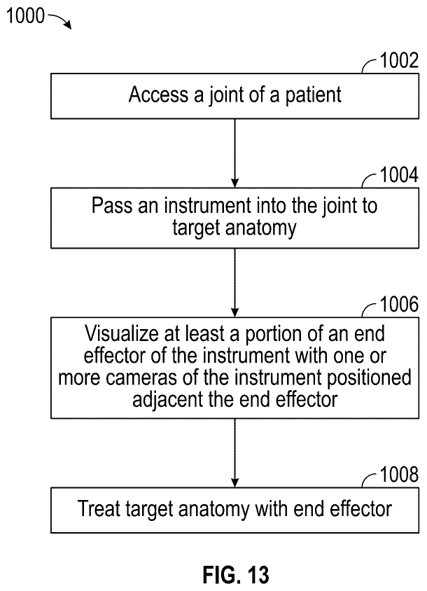

FIG. 13 illustrates a method 1000 for performing arthroscopic procedure such as a meniscectomy or other procedure. The method 1000 can include accessing a joint of a patient 1002. This accessing can be via a single access port that receives a shaft of an arthroscopic instrument. The method 1000 can include passing the arthroscopic instrument into the joint to a torn meniscus of the joint 1004 or to another anatomical structure. The method 1000 can include visualizing at least a portion of an end effector of the arthroscopic instrument with one or more cameras coupled to the arthroscopic instrument adjacent the end effector 1006. The method 1000 can include treating the meniscus with the end effector 1008 such as by performing mechanical cutting, RF ablation or RF coagulation.

The method 1000 can optionally include treating the meniscus with the end effector includes mechanically cutting the meniscus with actuation of a moveable jaw component. The treating the meniscus with the end effector can include providing RF energy to the meniscus. The treating the meniscus with the RF energy can include moving a position of an electrode with respect to a stationary portion of the end effector. Treating the meniscus the end effector can include providing a fluid inflow and outflow to the end effector. The visualizing can include illuminating the at least the portion of the end effector with a light source coupled to the arthroscopic instrument adjacent the end effector. The one or more cameras can be positioned with a field of view that includes the at least the portion of the end effector and a target tissue including the meniscus (or other target anatomical structure). The method 1000 can include moving the one or more cameras from a first position with a first field of view that includes the at least the portion of the end effector to a second position with a second field of view that differs from the first field of view. The method 1000 can include orienting the end effector and the one or more cameras prior to visualizing and treating.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The term “substantially”, “generally” or “about” mean within 15% of the value provided. The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. An arthroscopic instrument comprising:
an elongate shaft extending along a longitudinal axis;
at least one of a hub or a handpiece coupled to the elongate shaft;
an end effector coupled to a distal end of the elongate shaft and having a moveable jaw component; and
one or more cameras coupled to the elongate shaft and positioned adjacent the end effector.

2. The arthroscopic instrument of claim 1, wherein the moveable jaw component includes an electrode at a distal end portion thereof.

3. The arthroscopic instrument of claim 1, further comprising an electrode coupled to a suction tube of the end effector.

4. The arthroscopic instrument of claim 3, wherein the suction tube is configured to be moveable proximal-distal along the end effector including being configured to be moveable to a position at or distal of a distal tip of the end effector.

5. The arthroscopic instrument of claim 1, wherein the one or more cameras are part of a visualization assembly that is configured to be moveable from a first position that has a first field of view including at least a portion of the end effector to a second position with a second field of view that differs from the first field of view.

6. The arthroscopic instrument of claim 5, wherein the visualization assembly is at least one of rotatable relative to the end effector or extendible and retractable relative to the end effector.

7. The arthroscopic instrument of claim 6, wherein the visualization assembly includes a light source.

8. The arthroscopic instrument of claim 1, wherein the shaft includes one or more ports configured for inflow or outflow of a fluid proximal of the end effector.

9. The arthroscopic instrument of claim 1, wherein the one or more cameras and the end effector are angled relative to the longitudinal axis of the elongate shaft.

10. The arthroscopic instrument of claim 9, further comprising an articular joint connecting the one or more cameras and the end effector to the elongate shaft, wherein the articular joint is configured to move the one or more cameras and the end effector relative to the longitudinal axis.

11. An arthroscopic instrument for performing a meniscectomy comprising:
an elongate shaft extending along a longitudinal axis;
at least one of a hub or a handpiece coupled to the elongate shaft;
an end effector coupled to a distal end of the elongate shaft and having a moveable jaw component; and
a visualization assembly positioned adjacent the end effector and including one or more cameras with a field of view that includes at least a portion of the end effector and a target tissue including a torn meniscus.

12. The arthroscopic instrument of claim 11, wherein the end effector includes an electrode for delivering radiofrequency therapy, wherein the electrode is coupled to one of: the moveable jaw component, a stationary jaw component or a suction tube.

13. The arthroscopic instrument of claim 12, wherein the suction tube is configured to be moveable proximal-distal along the end effector including being configured to be moveable to a position at or distal of a distal tip of the end effector.

14. The arthroscopic instrument of claim 11, wherein the visualization assembly that is configured to be moveable from a first position with the field of view to a second position with a second field of view that differs from the first field of view.

15. The arthroscopic instrument of claim 14, wherein the visualization assembly is at least one of rotatable relative to the end effector or extendible and retractable relative to the end effector.

16. The arthroscopic instrument of claim 14, wherein the visualization assembly includes a light source.

17. The arthroscopic instrument of claim 11, wherein the shaft includes one or more ports configured for inflow or outflow of a fluid proximal of the end effector.

18. The arthroscopic instrument of claim 11, wherein the visualization assembly and the end effector are one of: angled relative to the longitudinal axis of the elongate shaft or rotatable relative to the longitudinal axis of the elongate shaft.

19. A method for performing arthroscopic meniscectomy comprising:
accessing a joint of a patient;
passing an arthroscopic instrument into the joint to a torn meniscus of the joint;
visualizing at least a portion of an end effector of the arthroscopic instrument with one or more cameras coupled to the arthroscopic instrument adjacent the end effector; and
treating the meniscus with the end effector.

20. The method of claim 19, wherein treating the meniscus with the end effector includes mechanically cutting the meniscus with actuation of a moveable jaw component, wherein treating the meniscus with the end effector includes providing radiofrequency energy to the meniscus, and wherein the treating the meniscus with the radiofrequency energy includes moving a position of an electrode with respect to a stationary portion of the end effector.
